(19) United States
(12) Patent Application Publication
(10) Pub. No.: US 2026/0018790 A1
(43) Pub. Date: Jan. 15, 2026

(54) COPLANAR WAVEGUIDE-FED CONFORMAL SPACE-FILLING ANTENNA BASED INGESTIBLE CAPSULE ENDOSCOPE SYSTEM

(71) Applicants: Vikrant Kaim, New Delhi (IN); Sumon Modak, Warangal (IN); Aijaz Mehdi Zaidi, Jalandhar (IN); Ladislau Matekovits, Turin (IT); Binod Kumar Kanaujia, Jalandhar (IN)

(72) Inventors: Vikrant Kaim, New Delhi (IN); Sumon Modak, Warangal (IN); Aijaz Mehdi Zaidi, Jalandhar (IN); Ladislau Matekovits, Turin (IT); Binod Kumar Kanaujia, Jalandhar (IN)

(21) Appl. No.: 19/335,107

(22) Filed: Sep. 22, 2025

(30) Foreign Application Priority Data
Apr. 28, 2025 (IN) 202511041106

(51) Int. Cl. H01Q 5/30; A61B 1/00; A61B 1/04; A61B 5/00; A61B 5/01; H01Q 1/52; H01Q 9/04; H01Q 9/26

(52) U.S. Cl. CPC H01Q 5/30; A61B 1/00016; A61B 1/041; A61B 5/01; A61B 5/6861; H01Q 1/526; H01Q 9/045; H01Q 9/26

(57) ABSTRACT

The present disclosure generally relates to a compact, conformal space-filling antenna system integrated within an ingestible capsule endoscope. The system utilizes a coplanar waveguide (CPW)-fed antenna, featuring a notched ground plane, a meandering radiating patch, and a parasitic patch, both designed in a serpentine pattern. This configuration enables operation at two distinct frequency bands: approximately 0.915 GHz with a 150 MHz bandwidth and 2.4 GHz with a 350 MHz bandwidth. The capsule houses a central processing unit for data transmission and sleep/wake-up functionality, powered by an internal battery. The antenna's conformal design maximizes space utilization within the cylindrical capsule, ensuring efficient wireless communication for medical diagnostic applications.

### CROSS-REFERENCE TO RELATED APPLICATION

This application claims the benefit of and priority to India patent application Ser. No. 202511041106 filed Apr. 28, 2025, the contents of which being incorporated by reference in its entirety herein.

### TECHNICAL FIELD

The present disclosure pertains to the design and implementation of wireless communication systems for medical diagnostic devices, specifically focusing on miniaturized antennas integrated within ingestible capsule endoscopes. More particularly, it relates to conformal space-filling antennas, utilizing coplanar waveguide (CPW) feeding techniques, optimized for multi-band operation within the constraints of a capsule's limited internal volume.

### BACKGROUND

The field of ingestible medical devices (IMDs) has revolutionized medical diagnostics and therapeutics, with wireless capsule endoscopy (WCE) being a prominent application. A critical component of these IMDs is the antenna, which faces significant design challenges due to stringent requirements for bandwidth, size, safety, and efficiency. Existing ingestible antenna designs often operate in single, dual, or multiple frequency bands, but suffer from limitations such as structural complexity, large physical dimensions, restricted bandwidth, and diminished gain.

Single-band designs, like meandered or rectangular loop patch antennas at 433 Mhz or 2.45 Ghz, and differential-fed conformal antennas at 915 MHz, have been reported. Similarly, dual-band designs using circularly polarized meander-shaped antennas at 915 MHz and 2450 MHz, or orthogonal strip and L-strip configurations at 1.4 GHz and 2.45 Ghz, have been explored. Tri-band and multi-band antennas, including conformal loop antennas operating at 403 MHz, 915 MHz, and 2.4 GHz, and inverted F-shaped antennas for MedRadio and ISM bands, have also been investigated. Multiport antennas, including meander-shaped and MIMO configurations, have been used, but increase bulk.

Despite these efforts, challenges remain in achieving compact, efficient, and multi-band antenna designs suitable for WCE. This disclosure addresses these limitations by introducing a novel capsule antenna system utilizing an interdigital patch structure, a parasitic radiator, and a notched ground plane, enabling operation at 0.915 GHz and 2.4 GHz with improved bandwidth and compactness, while also addressing SAR and communication link performance.

### BRIEF SUMMARY

The present disclosure seeks to provide a real-time functional CPW-fed compact (21×11 mm sq.) dual ISM band space-filling conformal ingestible capsule endoscope system, validated by wireless temperature monitoring of Wistar rat. Capsule integrated comb-shaped structures as space-filling patches and notched ground in the form of a loop are designed as a radiating patch in a muscle-mimicking cubic model for wireless communication. Surface current distribution on the ground resembles to that of the folded λg/2 dipole with antenna modeat both 0.915 (0.9-1.05 GHz) and 2.4 GHz (2.2-2.55 GHz) with quasi-omnidirectional radiation patterns having total gain of −15.1 dBi and −15.3 dBi, respectively. Electromagnetic interference (EMI) is also evaluated by wrapping different shielding layers between the antenna and the enclosed packaged battery. In the simulation, the proposed capsule's performance (S11, gain, 2D patterns) is further evaluated inside an anatomical human torso model in HFSS and a rat model in Sim4Life. As per ITU-R RS.1346 guidelines, the dosimetry study in rat and torso model qualifies the capsule device as suitable and safe for human use. A capsule device is fabricated for experimental validation, and performance is measured inside a muscle-imitating phantom, minced pork, and Wistar rat in the laboratory. The measured performance is satisfactory and acceptable at 0.915 and 2.4 GHz. Finally, using a software-defined radio (SDR) module and sensor, the temperature of the Wistar rat is monitored wirelessly when the data transfer rate is 1 Mbps and 25 Mbps. Moreover, this is the first ingestible capsule-based in-vivo study using Wistar rat, which can handle seamless wireless communication even beyond a 3 m distance away from the capsule.

In an embodiment, a capsule antenna system is disclosed. The system includes a cylindrical capsule shell fabricated of a biocompatible acrylic material having a thickness of 0.5 millimeter, a relative permittivity of 3.0, and a loss tangent of 0.001, the capsule shell having an outer height of 21 millimeters and an outer diameter of 11 millimeters.

In an embodiment, an internal cavity within the capsule shell is configured to house a battery and circuit components, the battery being modeled as a perfect electric conductor.

In an embodiment, a planar antenna structure is fabricated on a substrate of dimensions 9 millimeters by 31 millimeters, the substrate having a thickness of 0.254 millimeter, a relative permittivity of 2.2, and a loss tangent of 0.008.

The planar antenna structure comprising a coplanar waveguide (CPW) feed system, a radiating patch, and ground strips positioned on a same plane. The planar antenna structure being wrapped onto an inner cylindrical surface of the capsule shell to form a conformal antenna, wherein the substrate acts as a dielectric shield between the radiating patch and the battery.

In an embodiment, a multilayer dielectric configuration surrounding the antenna structure comprising the substrate as a first dielectric layer, the acrylic capsule shell as a second dielectric layer.

In an embodiment, an external medium as a third dielectric layer, the combined layers defining an effective permittivity influencing current distribution and resonant behavior, wherein the radiating patch, the ground strips, and the feed system are dimensioned and arranged such that current distribution along the antenna structure follows half guided wavelength paths with patch currents traveling opposite to ground currents.

An object of the present disclosure is to develop a simple and compact coplanar waveguide (CPW) fed dual-band capsule antenna for Wireless Capsule Endoscopy (WCE).

Another object of the present disclosure is to achieve operation within the industrial, scientific, and medical (ISM) bands, specifically at 0.915 (0.9-1.05 Ghz) and 2.4 GHz (2.2-2.55 Ghz).

Another object of the present disclosure is to design an antenna configuration incorporating an interdigital structure and a truncated ground plane.

Another object of the present disclosure is to enhance antenna performance by introducing a parasitic radiator, enabling operation within the desired frequency bands.

Another object of the present disclosure is to evaluate the antenna's functionality using a homogeneous human body model and a Wistar rat model.

Another object of the present disclosure is to experimentally validate the antenna's performance in conformal configurations using a solid muscle-imitating phantom and minced pork.

Another object of the present disclosure is to attain gain values of approximately −15.1 dBi at 0.915 GHz and −15.3 dBi at 2.4 GHz.

Another object of the present disclosure is to assess the communication link and specific absorption rate (SAR) using a software-defined radio with the capsule antenna implanted in the stomach of a Wistar rat and a realistic torso phantom.

Yet another object of the present disclosure is to deliver an expeditious and cost-effective safe antenna solution for biotelemetry applications in ingestible medical devices.

To further clarify the advantages and features of the present disclosure, a more particular description of the disclosure will be rendered by reference to specific embodiments thereof, which are illustrated in the appended drawings. It is appreciated that these drawings depict only typical embodiments of the disclosure and are therefore not to be considered limiting of its scope. The disclosure will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1(a) illustrates proposed antenna conformal to capsule shell;

FIG. 1(b) illustrates Cross-sectional profile of an implantable antenna showcasing three dielectric layers in surrounding. Rogers as substrate, and acrylic as superstrate;

FIG. 2 illustrates Design steps of the coplanar conformal antenna (shown in planar form) Antenna-3 is the final proposed design;

FIG. 3(a) illustrates Surface current distribution on the radiator and ground of Antenna-1 (flat form);

FIG. 3(b) illustrates Surface current distribution on the radiator and ground of Antenna-1 (conformal form);

FIG. 3(c) illustrates Surface current distribution on the radiator and ground of Antenna-2 (conformal form);

FIG. 3(d) illustrates Surface current distribution on the radiator and ground of Antenna-3 (conformal form);

FIG. 4(a) illustrates Simulated performance comparison of the capsule antenna design stages in the conformal form: Reflection coefficient (|S11|);

FIG. 4(b) illustrates Simulated performance comparison of the capsule antenna design stages in the conformal form: Total gain;

FIG. 5(a) illustrates Simulated performance comparisons (Case 1 to Case 3): Schematic for three cases;

FIG. 5(b) illustrates Simulated performance comparisons (Case 1 to Case 3): |S11|;

FIG. 5(c) illustrates Simulated performance comparisons (Case 1 to Case 3): Total gain;

FIG. 6(a) illustrates Plot of E-field distribution (scale: V/m) when the observation line passes through the battery,

FIG. 6(b) illustrates Plot of E-field distribution (scale: V/m) when the observation line passes through the battery,

FIG. 6(c) illustrates Plot of E-field distribution (scale: V/m) when the observation line passes off the battery,

FIG. 6(d) illustrates Plot of E-field distribution (scale: V/m) when the observation line passes off the battery,

FIG. 6(e) illustrates S11 plots in the presence of battery;

FIG. 6(f) illustrates S11 plots in the absence of battery;

FIG. 7(a) illustrates Performance comparisons through varying the thickness of the capsule shell and materials: |S11|;

FIG. 7(b) illustrates Performance comparisons through varying the thickness of the capsule shell and materials: Gain;

FIG. 7(c) illustrates Performance comparisons through varying the thickness of the capsule shell and materials: |S11|;

FIG. 7(d) illustrates Performance comparisons through varying the thickness of the capsule shell and materials: Gain;

FIG. 8(a) illustrates Simulation models and comparison of results: Ella Human anatomical model;

FIG. 8(b) illustrates Simulation models and comparison of results: Rat model;

FIG. 8(c) illustrates Simulation models and comparison of results: |S11| gains;

FIG. 9(a) illustrates fabricated conformal antenna in flat form;

FIG. 9(b) illustrates 3D printed capsule with wrapped antenna and battery inside;

FIG. 10(a) illustrates Comparison of simulated and measured performance of the fabricated capsule prototype: Reflection coefficient (|S11|);

FIG. 10(b) illustrates Comparison of simulated and measured performance of the fabricated capsule prototype: Total Gain;

FIG. 11 illustrates Simulated far-field 3D radiation patterns are obtained with the capsule positioned in the XY and XZ planes inside the small intestine of the human Ella model and the stomach of the rat model;

FIG. 12 illustrates Comparison of simulated and measured radiation patterns in muscle phantom, minced pork, stomach, small intestine (SI), large intestine (LI);

FIG. 13 illustrates corneal view of averaged SAR distribution in Ella human anatomical human over 1 g of tissue standards;

FIG. 14 illustrates Coronal and Sagittal view of averaged SAR distribution in anatomical rat model over 1 g of tissue standards;

FIG. 15(a) illustrates Simulated (calculated) and experimental: link budget at different data rates with varying distances between Tx and Rx, at 0.915 GHz and 2.4 GHz;

FIG. 15(b) illustrates Simulated (calculated) and experimental: received power at different data rates with varying distances between Tx and Rx, at 0.915 GHz and 2.4 GHz;

FIG. 16 illustrates a table depicting comparison of the proposed capsule antenna system with state-of-the-art;

FIG. 17 illustrates a table depicting performance comparison of the three cases;

FIG. 18 illustrates a table depicting simulated performance behavior on different phantoms models;

FIG. 19 illustrates a table depicting link budget parameters for the proposed capsule antenna;

FIG. 20 illustrates a table depicting in-vivo experimental performance comparison on rat; and

FIG. 21 illustrates a block diagram of a capsule antenna system in accordance with an embodiment of the present disclosure.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail concerning the accompanying drawings.

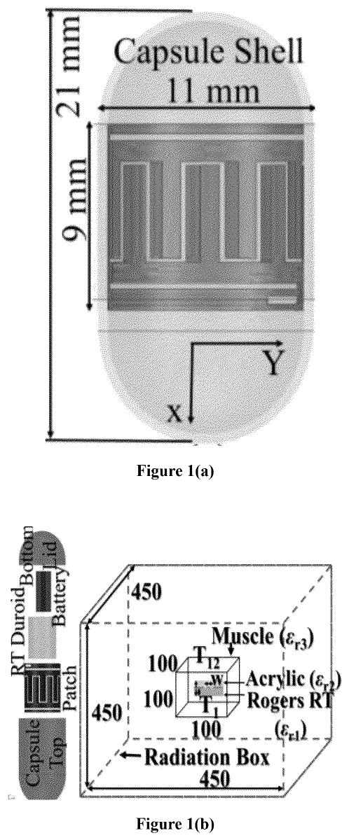

Referring to FIG. 1, (a) Proposed antenna conformal to capsule shell, (b) Cross-sectional profile of an implantable antenna showcasing three dielectric layers in surrounding. Rogers as substrate, and acrylic as superstrate is illustrated in accordance with an embodiment of the present disclosure.

### Architecture of the Capsule Antenna

FIG. 1 illustrates the geometric configuration of a dual-band conformal capsule antenna system integral to the functionality of medical capsule endoscopy systems for visual inspection and data collection from within the GI tract. The cylindrical capsule structure shown in FIG. 1(a) is made from the biocompatible 3D printed, 0.5 mm thick acrylic material (εr=3.0, tanδ=0.001), and having an outer height and diameter of 21 mm and 11 mm, respectively. The antenna is designed on Rogers 5880 substrate, characterized by dimensions 9×31 mm2, which exhibits specific electrical properties with a relative permittivity and loss tangent (εr=2.2, tanδ=0.008) accompanied by a thickness, 0.254 mm. This coplanar antenna configuration employs a CPW feeding mechanism, featuring a notched ground plane integrated along the periphery of the radiating structure on the same plane. The arrangement of the antenna and other circuitry (packaged battery) components has been strategically done inside the capsule shell. Moreover, in practical applications, antennas are often integrated into cylindrical capsule structures that house various electronic components, including batteries, sensor circuits, and other relevant elements. The battery inside the capsule is the power source to run the embedded circuit and, if required, can be recharged wirelessly after its duty cycle gets exhausted. However, the conductive nature of these internal components can potentially impact the antenna's performance. Interestingly, due to the coplanar antenna configuration, the Rogers 5880 substrate also acts as a dielectric shield between the radiator and the battery, which is modeled as PEC. This arrangement provides an add-on advantage as the inner hollow space is intentionally reserved to serve as the housing for electronic components.

### Operating Mechanism of the Capsule Antenna

Implantable antennas have to function within the human body, where the surrounding tissues possess high dielectric permittivity and conductivity. In addition, such antennas must also be encapsulated by superstrate to avoid short circuits with the neighboring conductive tissues. As multiple dielectric layers above the antenna influence fringing fields; as a result, the antenna's effective relative permittivity (εeff) will increase compared to its operation in free space. Therefore, in the context of the proposed work, the effective permittivity is calculated based on a model that contains three separate layers in the surrounding of an antenna: substrate, superstrate, and muscle tissues (εr1=2.2, εr2=3, εr3=54.9); as shown in FIG. 1(b). RT duroid (substrate) and acrylic (superstrate) have thickness T1=0.254 mm, and T2=0.5 mm, respectively, where, T12=(T1+T2)=0.754 mm (copper thickness neglected for simplification). For a microstrip line of width w, generalized closed form expressions are used to calculate εeff for (wT [symbol] 1). Based on the calculated effective permittivity (εeff), the design evaluation of the proposed capsule antenna is carried out.

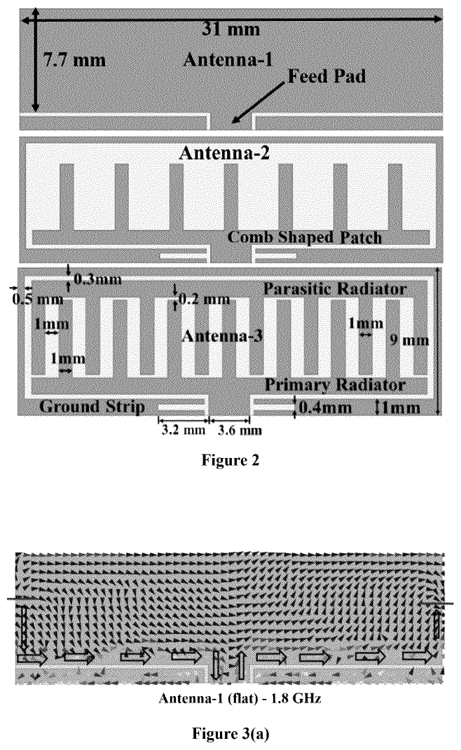

FIG. 2 illustrates Design steps of the coplanar conformal antenna (shown in planar form) Antenna-3 is the final proposed design. FIG. 2 shows the progressive design evolution (Antenna-1 to Antenna-3) of the capsule antenna. Notably, the design stages illustrated in FIG. 2 depict the antenna claddings in a planar configuration for ease of understanding the structure.

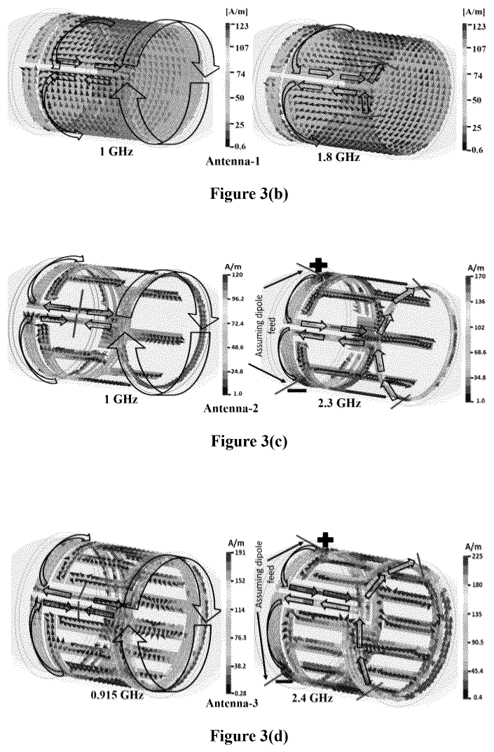

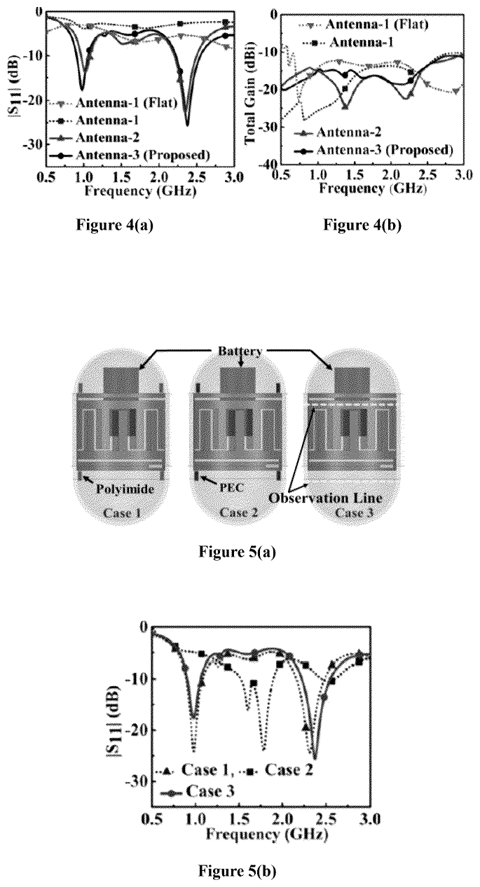

FIG. 3 illustrates Surface current distribution on the radiator and ground of Antenna-1 (flat & conformal form) and Antenna-2, 3 (conformal form). Thick solid lines in red depict minima. However, during the simulation, each stage was developed in conformal form by analyzing the surface current distribution, as shown in FIG. 3. Prior to wrapping the antenna, to restrict the dimensions suitable to capsule endoscopy, a rectangular patch element having dimensions of l=7.7 mm and w=31 mm with coplanar ground strips is considered and simulated in planar form as per the simulation model shown in FIG. 1(b). Antenna-1 in planar configuration supports simulated resonant frequency at 1.8 GHz with poor impedance match where, |S11|=−6 dB (see FIG. 4a).

$$\frac{c}{{λ}_{g} \sqrt{{ε}_{reff}}} = \frac{c}{2 L \sqrt{{ε}_{reff}}} {λ}_{g} = {λ}_{o} / \sqrt{{ε}_{reff}} \qquad ( 1 )$$

In the current distribution on the surface of planar Antenna-1, the current resembles that of the folded half-wavelength dipole antenna, where, λg and λo denotes guided and free-space wavelength, respectively. From minima (solid red line) to minima, the total path length (L) is 44.9 mm=λg/2. Moreover, current on ground strips travel in a direction opposite to that of the patch, as it should be. Using (1), frequency (fr) is calculated to be 2.15 GHz where, εeff=2.409, L=44.9 mm, and c=3×108 m/s. It is noteworthy that the gap between the patch and ground strips exhibits higher sensitivity, which necessitated a comprehensive parametric study during the design phase of Antenna-1. The analysis considered two strategies, variation of the patch width and modification of the ground strip. This revealed that adjusting the gap and strip width extends the operational bandwidth beyond 4 GHz. However, the corresponding analysis has been excluded here for brevity. Since the primary objective is to achieve dual ISM band operation at 0.915 and 2.4 GHz, an optimized gap of 0.3 mm was selected as the most suitable configuration. Accordingly, the subsequent discussion is restricted to frequencies below 3 GHz to address the desired application requirements.

FIG. 4 illustrates Simulated performance comparison of the capsule antenna design stages in the conformal form: (a) Reflection coefficient (|S11|), (b) Total gain. Since the goal is to design a capsule antenna for operation within the GI tract, the simulation methodology involves the insertion of the capsule antenna at the center position of a homogenous muscle phantom of size 100×100×100 mm3, which encompasses electrical properties at the lowest frequency band of 0.915 Ghz, where permittivity, εr=54.9, and conductivity, σ=0.948 S/m. Additionally, to minimize boundary reflections and ensure accurate far-field calculations, radiation boundaries are defined at 450×450×450 mm3, exceeding λₒ/4 at 0.915 GHz. Further, the design of the capsule antenna was initiated by wrapping the planar Antenna-1 around the inner wall of the cylindrical capsule structure, allowing the Antenna-1 to conform to the capsule's curvature. Conformal Antenna-1 is showing resonance at 1 Ghz and 1.8 GHz with a poor impedance match (see FIG. 4(a)). At 1 GHz and 1.8 GHz in FIG. 3(b), the current start from the feed and travels along the path shown by arrows, then return to the feed, covering a total path length of 83.6 mm and 56.4 mm, respectively. These lengths are λg/2 as the current distribution on the path lengths resembles that of the folded half-wavelength dipole antenna. The calculated resonant frequencies are 1.15 GHz and 1.71 Ghz, respectively. Further, in the design of Antenna-2, the ground strips are notched near the feed and extend to curl the substrate at the edges to get enough electrical length for the surface current so that the ground perimeter marked with arrows (in FIG. 3(c)) could shift the resonant frequencies to 0.915 and 2.4 GHz. The rectangular patch which is modified to the comb-shape structure, and the notched ground strips are used for impedance matching. This straightforward design enabled the Antenna-2 to operate at dual frequency bands of 1 Ghz (0.95-1.08 Ghz) and 2.3 Ghz (2.2-2.5 Ghz) exhibiting effective impedance matching with fractional bandwidths exceeding 13% in both bands (see FIG. 4(a)). As evident in FIG. 3(c), since coplanar feed port is providing a direct electrical connection between notched ground strips with patch's feed pad (in FIG. 2); at 1 GHz current start from the feed and travel along the path shown by arrows which resemble to that of the folded half-wavelength dipole antenna. Before returning to the feed, in simulation the current covers a path length of 80 mm, which is λg/2 at 1 GHz whereas, calculated fr is 1.21 GHz. At 2.3 Ghz, current start from the assumed dipole feed location on the ground, as shown in FIG. 3(c), and covers a total path length of 46 mm, which is λg/2 at 2.3 Ghz, resembling that of the folded half-wavelength dipole antenna. The simulated fr is close to the calculated fr of 2.1 Ghz.

Although a dual-band response is achieved in Antenna-2, resonances are fine-tuned to the proposed ISM bands (0.915 Ghz and 2.4 Ghz) by introducing another inverted comb-shaped structure as a parasitic stub in a final proposed Antenna-3, which makes the input impedance of the feed port purely resistive at the desired frequencies. Since surface current distribution in Antenna-3 resembles that of Antenna-2 (see FIG. 3(d)); as a result, the lower operating frequency band shifted to 0.915 GHz while the higher frequency band shifted to 2.4 GHz with an impedance bandwidth of 150 Mhz (0.9-1.05 Ghz) and 350 Mhz (2.2-2.55 GHz). Although, the achieved impedance bandwidths, corresponding to fractional bandwidths of 16.3% and 14.5% are not exceptionally wide, however, they are sufficiently optimized to support practical data rates, tailored for the proposed applications. Additionally, a notable enhancement and stability in gain is observed (see FIG. 4(b)) due to the increment in radiation aperture area. Noteworthy, while designing, it is observed that different bending angles change the amount of field coupled between parasitically loaded comb-shaped patch and ground strips. Due to this, the current path length varies and, thus, changes the resonant frequency.

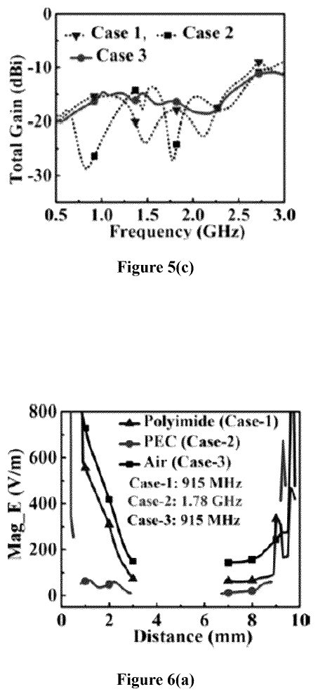

FIG. 5 illustrates Simulated performance comparisons (Case 1 to Case 3): (a) Schematic for three cases (b) |S11|, (c) Total gain.

### Effects of Electromagnetic Interference

This section presents a detailed study of the impact of the fields radiated from the rolled antenna on the surrounding electronic circuitry (battery) within the cylindrical capsule system. The investigation evaluates electromagnetic interference (EMI) by wrapping a shielding layer between the antenna and the enclosed packaged battery, with height=9 mm and diameter=7 mm inside the cylindrical capsule under three cases. Case-1: when a 0.05 mm thick dielectric biocompatible polyimide layer (εr=4.3, tanδ=0.008) is used for shielding, Case-2: when a PEC layer of 0.03 mm is used for shielding, Case-3: when shielding layer is not used (see FIG. 5(a)); and this case also resembles the final proposed design configuration as explained in design evolution. FIGS. 5(b) and 5(c) show the performance comparison in terms of S11 and gain for the three cases, respectively, which are summarized in Table II. It is observed that the lossy dielectric layer (polyimide) in case-1 poses minimal detuning in the results. Whereas a PEC layer in case-2 merges both the resonant frequencies and shift closer to each other and deteriorates the gain. Noted, it is also observed that the PEC sheet (in case-2) reflects most of the fields (as Etan=0 on the PEC sheet), which interferes with the fields radiating from the antenna aggressively outside the capsule, and thus, the gain is compromised. This could be the possible reason for the much lower gain (−26.3 dBi) at 2.4 GHz compared to-15.1 dBi gain at 0.915 GHz in Case-2.

From the aforementioned analysis, it can be concluded that in the proposed capsule design, having an extra dielectric layer such as polyimide can be useful as a shield, as it does not influence the results. But, to make further space available for the electronics inside the capsule, authors choose to get rid of the extra shield by selecting a coplanar space-filling antenna design where the substrate (RT duroid) itself acts as a shield layer.

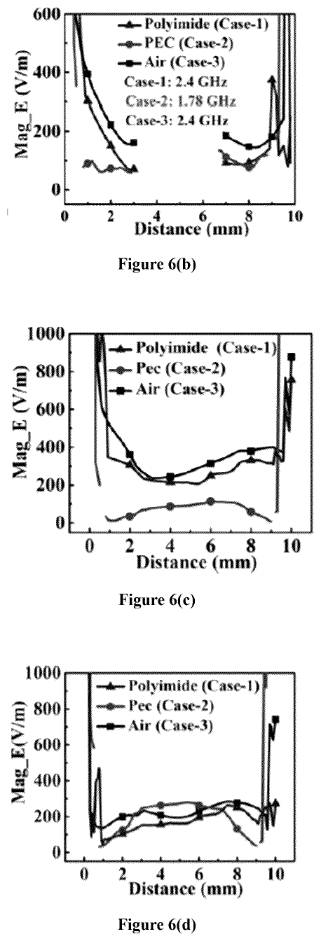

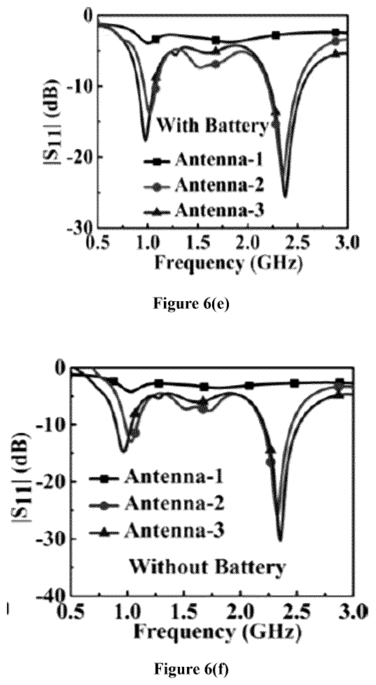

FIG. 6 illustrates Plots of E-field distribution (scale: V/m), (a-b) when the observation line passes through the battery, (c-d) when the observation line passes off the battery, and (c-f) S11 plots in the presence and absence of battery. FIG. 6 shows the E-field distribution as a function of the distance between the opposite faces of the rolled antenna inside the capsule shell. The observation line where E-fields are taken (See FIG. 5(a) (Case 3), we have the battery, and as evident from the results at both lower and higher frequencies, shown in FIG. 6(a-b), there is no strong coupling between the battery's surface and the rolled antenna. For the case of air, fields extended a bit. However, we don't have any field for all the cases after 3 mm from the antenna's surface. This is due to the battery's presence. Furthermore, field distribution is also shown in FIG. 6(c-d) along the observation line at the other end of the rolled antenna where the battery is not present. Results show some weak coupling in all the cases after 3 mm from the antenna's surface. Moreover, FIG. 6(e-f) illustrates that, even in the absence of a battery, the resonant frequencies and impedance matching remain largely stable, with only a slight shift to 0.89 GHz and 2.35 Ghz, corresponding to fractional bandwidths of 15.6% and 12.7%, respectively. Therefore, the battery is not enhancing the field coupling, and thus, there is no capacitive loading due to such weak coupled fields; hence, the resonance frequency should strictly follow the length of the current paths with a proper effective dielectric constant. Furthermore, a complementary study is conducted to systematically evaluate the electromagnetic performances of the capsule antenna. The first strategy appraises the influence of varying capsule wall thicknesses using acrylic, while the second investigates alternative biocompatible materials (PEEK (Polyether Ether Ketone), PLA (Polylactic Acid), and silicone elastomers) at the original wall thickness. Remarkably, the simulations were performed in a muscle-equivalent phantom to mimic realistic tissue interaction, using dielectric properties from PEEK and silicone elastomers.

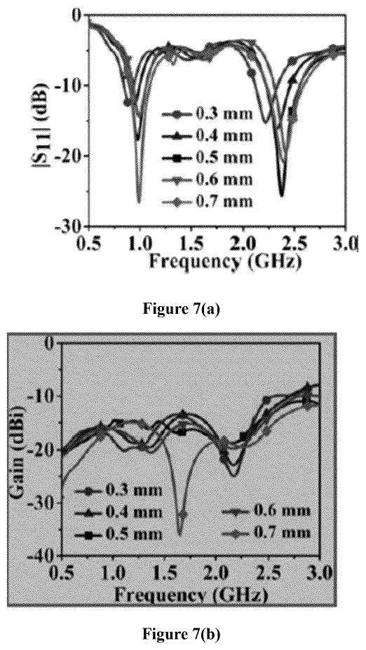

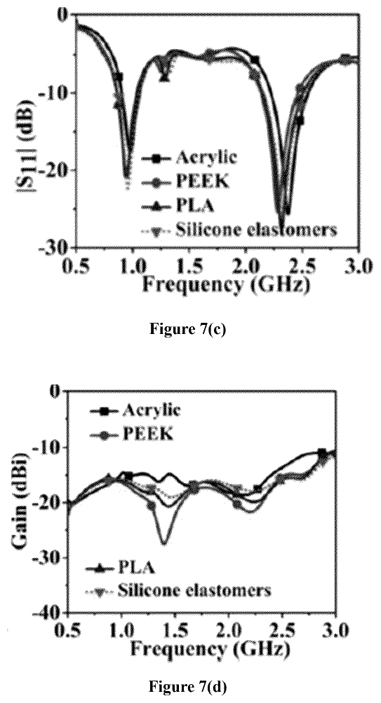

FIG. 7 illustrates (Performance comparisons through varying the thickness of the capsule shell and materials (a)-(c) |S11|, (b)-(d) Gain. FIG. 7(a) demonstrates the influence of capsule wall thickness variation. This reveals that on reducing the thickness causes a downward frequency shift, with the minimum thickness of 0.3 mm yielding resonances at 0.89 and 2.2 GHz. However, this shift degrades impedance matching, and dipping the fractional bandwidths to 11.2% and 9.9%.

In contrast, at 0.7 mm wall thickness, the antenna shows an upward resonance shift with improved impedance matching and broader bandwidths (20.2% and 15.6%), though the gain decreases to −17.1 dBi and −18.2 dBi respectively (see FIG. 7(b)). Likewise, the effect of different capsule materials is examined while keeping the wall thickness constant. As plotted in FIG. 7(c), all evaluated materials exhibit minimal downward frequency shifts, with silicone elastomer providing enhanced bandwidths (19.7% and 15.5%), and improved gain compared to PEEK and PLA materials (see FIG. 7(d)). These findings reveal that the variation in capsule wall thickness and substitution of capsule material induce a minimal detuning in resonance and gain, while maintaining stable operation within the target frequency band. Moreover, the suggested material offers a viable alternative for capsule fabrication in future implementations.

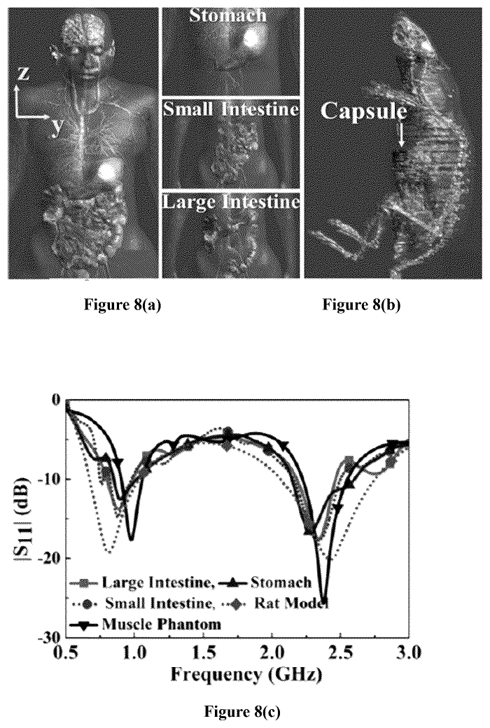

FIG. 8 illustrates Simulation models and comparison of results: (a) Ella Human anatomical model (b) Rat model, (c) |S11| gains.

### In-Vivo Measurement Using Wistar Rat

Following the initial simulations performed on a muscle phantom, we progressed to more realistic scenarios in simulation by positioning the capsule antenna within different tissue environments of a realistic Ella human anatomical model (Yoon-sun, 26 year old Women) and the stomach of a rat model before assessing its practical efficacy (See FIG. 8(a-b). These simulations are performed using a FDTD-based Sim4Life simulator. For each configuration, the capsule is placed at approximate depths of 32 mm (stomach), 50 mm (small intestine), and 65 mm (large intestine) from the nearest external body surface to systematically assess the antenna's electromagnetic behavior including its sensitivity to tissue loading, impedance mismatch arising from detuning effects, and variations in radiation performance. Since, the measurements are planned for in-vivo validation in a Wistar rat under medical supervision, simulation is also conducted using a rat model to evaluate the anticipated performance of the proposed ingestible capsule. The simulated (|S₁₁|) responses of the optimized capsule antenna in the muscle phantom, human anatomical model (with three implant locations), and rat model are illustrated in FIG. 8(c). These results reveal that the antenna consistently maintains broad impedance bandwidths exceeding approximately 16% and 11.5% across all tissue environments, while a slight downward shift in resonant frequency and minor impedance mismatch are also observed, along with a reduced peak gain of −22.7 dBi/−18.1 dBi within the small intestine, and to −21.06 dBi/−17.4 dBi and −20.01dBi/−16.2 dBi in the stomach and large intestine, which is not shown for brevity. In contrast the rat model exhibited resonant frequencies at 0.82 GHz and 2.42 GHz, maintaining wide impedance bandwidths and peak gains of −18.7 dBi and −17.6 dBi, respectively. The minor variations in resonant characteristics are likely due to differences in dielectric loading from anatomical and structural variations between human and rat models, along with the distinct meshing schemes used in HFSS and Sim4Life, which minimally alter the surface current distribution and thereby lead to negligible changes in the effective current path length. Nevertheless, the simulated results closely align with those of the homogeneous muscle phantom (resonant frequencies: 0.915 GHz and 2.4 GHz; peak gains: −15.1 dBi and −15.3 dBi), ensure that consistently covering the target bands for wireless capsule endoscopy. A detailed comparison of key metrics across all phantom models are summarized in Table III.

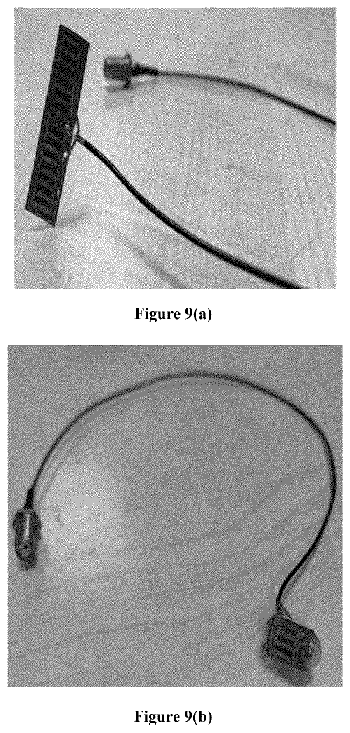

FIG. 9 illustrates (a) Fabricated conformal antenna in flat form, and (b) 3D printed capsule with wrapped antenna and battery inside. Further, the simulation is validated by measuring the proposed dual-band capsule antenna system's performance in the same environment (muscle phantom, minced pork, and Wistar rat). The proposed design of the conformal antenna is fabricated in planar form and subsequently integrated by conformally wrapping it around the inner wall of the battery-fitted 3D-printed capsule shell, as shown in FIG. 9(a-b). The metallic cylinder is modeled as a battery. The cylindrical capsule shell is made of acrylic, and the two end caps (domes) are made of transparent resin, which are affixed to the top and bottom of the cylindrical shell. A hole is drilled into the capsule cap to allow the coaxial cable to pass through and provide essential connectivity to the antenna feed port. Additionally, an adapter cable (SFT50-1) SMA male to SMA male is utilized to connect the 50-62 coaxial cable to the handheld two-port Keysight N9926A and Anritsu MS2038C vector network analyzer (VNA). To imitate the simulation conditions, the S-parameter (|S11|) of the proposed capsule is measured inside the gel-based muscle phantom and freshly butchered minced pork muscles covered by fat layer by embedding the prototype at 50 mm depth kept in a plastic container having dimensions 120×80×80 mm³. For the |S11| measurement inside the Wistar rat, the capsule is inserted inside the abdomen of the rat. Noteworthy, all the in-vivo testing was done under aseptic conditions as per the guidelines and approval of the Institutional Animal Ethics Committee (certificate no. 288/IAEC-1/2021). The measurements were carried out at the All India Institute of Medical Sciences (AIIMS), New Delhi, India. The 16-week-old male Wistar rat animal subjects were acclimatized to the animal house facility of AIIMS for 7 days. The subject was kept on fasting overnight before the day of measurement with access to only drinking water. On day-1 of the study, an aseptic workstation was prepared using 70% alcohol to spray the workstation and instrument trays. The operating table and instrument trays were covered with a sterile drape before the sterilized instruments were placed on the tray. Initially, the subject was anesthetized using Pentobarbitone (45 mg/Kg, i.p.) and mounted on the dissection table, and then the abdomen hair was shaved off. After cleaning the shaved region with antiseptic, an incision of 12 mm was made on the abdomen toward the left of the midline and the stomach. Further, an incision of 6 mm was made from the minor curvature of the pyloric region, and the proposed capsule, along with a coaxial cable, was inserted and adjusted inside the stomach. Lastly, the incision on the pyloric region was sutured using 10 mm soluble monofilament sterile surgical needle sutures, and the peritoneal cavity using non-absorbable monofilament surgical needle sutures. Neosporin powder was also sprinkled to avoid any infection. Finally, the data (|S11| and body temperature) was recorded in stable conditions.

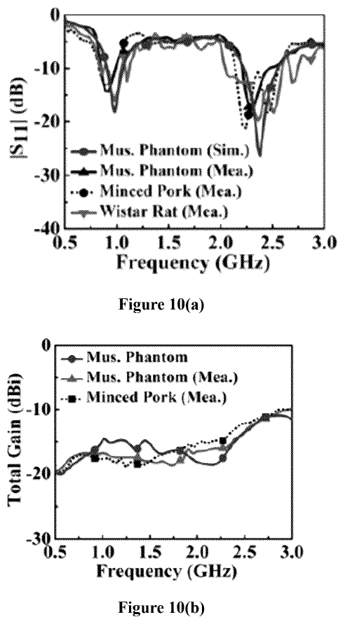

FIG. 10 illustrates Comparison of simulated and measured performance of the fabricated capsule prototype, (a) Reflection coefficient (|S11|), (b) Total Gain. The comparison of measured |S11| and gain under different surrounding conditions is illustrated in FIG. 10(a) and FIG. 10(b), respectively. Within the muscle phantom, the proposed capsule with the integrated battery demonstrates measured resonances at 0.91 Ghz and 2.36 Ghz, with impedance bandwidth of 15.3% and 11.4% achieving peak gain of −17.1 dBi and −14.6 dBi, respectively. In contrast, in the absence of battery, the measured impedance bandwidth reduces to 14.6% at 0.89 GHz and 10.5% at 2.28 GHz, accompanied by a noticeable degradation in peak gain to −18.2 dBi and −18.9 dBi, respectively. In minced pork, the measured resonating frequencies are 0.88 GHz and 2.31 GHz with a peak gain of −17.7 dBi and −13 dBi, respectively.

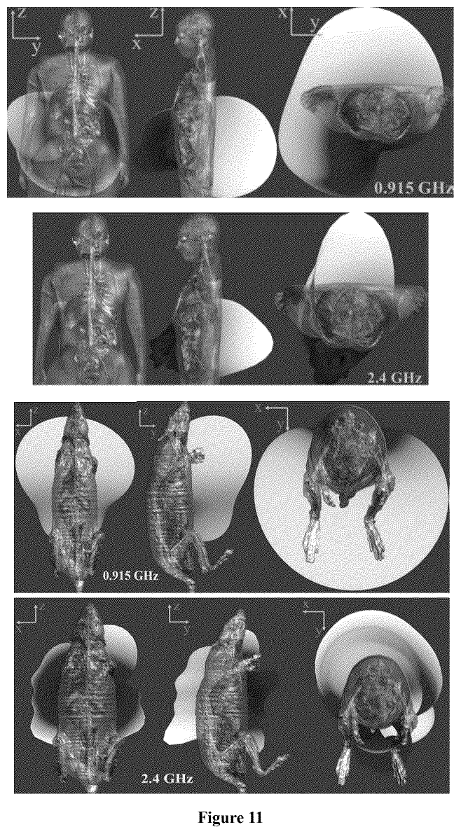

FIG. 11 illustrates Simulated far-field 3D radiation patterns are obtained with the capsule positioned in the XY and XZ planes inside the small intestine of the human Ella model and the stomach of the rat model. In rat animal subjects, the measured |S11| plot fluctuates near simulation results, having an overall good impedance match and, thus, shows consistency with simulated |S11| in the rat model. Despite having irregularities in measured results due to imperfect manual handling of the in-house facilities and inevitable path loss due to air pocket formation during implantation, largely, the measured performance of the capsule prototype that demonstrates good agreement with the simulation in the corresponding environs seems to be acceptable and convincing. During simulation in Sim4Life, the rat model does not consider body heat, interstitial fluid, and age factor effects that strongly influence the tissue's electrical properties. However, all such effects are inevitable during measurement inside live animal subjects. In WCE, the capsule experiences arbitrary orientation changes across the planes during GI transit. Hence, for reliable telemetry of imaging and diagnostic data to the external receiver, it is critical to characterize the antenna's far-field radiation patterns in all planes to ensure consistent omnidirectional coverage and polarization robustness. FIG. 11 shows the simulated patterns when the capsule is positioned in the XY and XZ plane inside the small intestine and stomach of the human Ella and rat the capsule antenna gives quasi omnidirectional radiation in elevation (XZ, XY) and azimuthal (YZ) plane in both the models, but in rat model, at 2.4 GHz patterns are relatively distorted due to structural and organ size variations in the rat model's GI tract and because the relative permittivity and conductivity of the stomach tissues are higher than the small intestines. Furthermore, the radiation performance was experimentally evaluated in a fully shielded anechoic chamber, with prior full two-port calibration of RF cables and the test setup (open, short, load, and through) to ensure measurement accuracy. For measurements, a standard horn served as the transmitter and the proposed capsule antenna as the receiver, aligned coaxially at a 3.1 m separation, with tests conducted using minced pork and a gel-based phantom to emulate realistic scenarios.

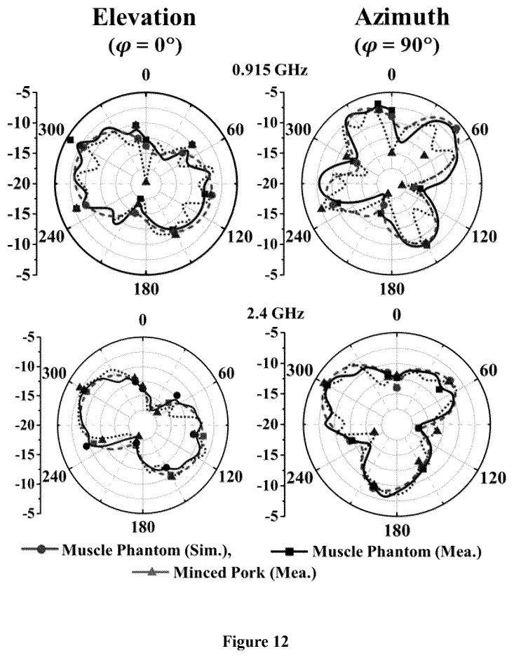

FIG. 12 illustrates Comparison of simulated and measured radiation patterns in muscle phantom, minced pork, stomach, small intestine (SI), large intestine (LI). FIG. 12 shows the comparison of simulated and measured far-field radiation patterns in elevation (φ=0□) and azimuthal (φ=90□) plane. Radiation patterns are measured inside an anechoic chamber where the capsule antenna is submerged in muscle phantom and minced pork. The measured counterparts imitate the simulation with acceptable distortion. At φ=0□, the main radiation beam is tilted to an angle φ=300□ whereas, at φ=90□. Similarly, when simulated within the anatomical body model, the radiation patterns exhibit increased distortion due to complex tissue heterogeneities. Nevertheless, the proposed antenna consistently demonstrates quasi-omnidirectional radiation characteristics across all scenarios at both 0.915 GHz and 2.4 GHz.

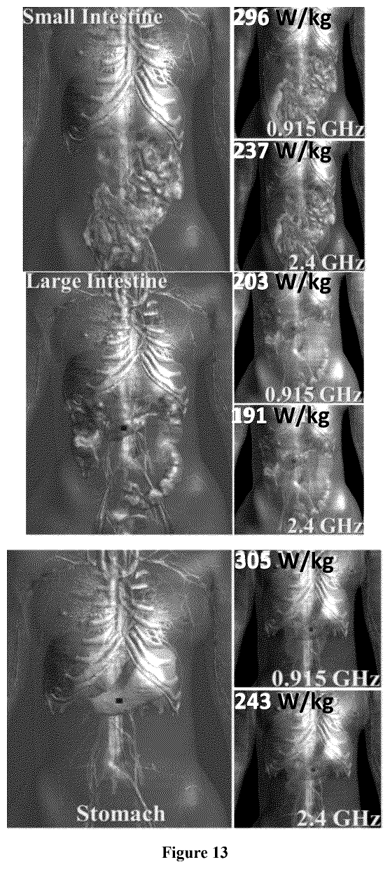

FIG. 13 illustrates corneal view of averaged SAR distribution in Ella human anatomical human over 1 g of tissue standards.

### Biological Tissues Safety Assessment

Given that the proposed antenna is designed for transferring real-time data to external devices in a wireless capsule endoscopy system, it is crucial to ensure electromagnetic (EM) radiation absorption safety. Excessive EM radiation can be harmful if it surpasses established safety limits. According to FCC and ICNIRP guidelines, the safety limits for EM waves are defined in terms of specific absorption rate (SAR). Specifically, for 1 g and 10 g of tissue, the SAR value must not exceed 1.6 W/Kg and 2 W/Kg. In this regard, the SAR value for 1 g of tissue mass is assessed at frequencies of 0.915 and 2.4 GHz. The SAR assessment is done in simulation by positioning the capsule antenna within the small intestines, large intestines, and stomach of a realistic human Ella model and the stomach of a rat model in Sim4Life. The antenna is excited by the reference input power of 1 W. From FIG. 13, results in the human model from corneal view indicate that the averaged SAR (ASAR) values over 1 g of tissue are 296 W/Kg and 237 W/Kg (SI), 203 W/Kg and 191 W/Kg (LI) and 305 W/Kg and 243 W/Kg (Stomach), while the results in the rat model from coronal and sagittal views indicate that the averaged SAR (ASAR) values over 1 g ASAR distribution in both the model shows that EM fields are largely absorbed and stretched out non-uniformly at 0.915 GHz but squeeze uniformly at 2.4 GHz, surrounding the capsule only. In order to comply with FCC and ICNIRP guidelines, the SAR due to the proposed capsule device can be maintained at a lower than the threshold value. The highest permissible input power to the capsule antenna must not exceed 0.0052 W (7.16 dBm) and 0.0065 W (8.13 dBm) corresponding to the highest simulated SAR values observed for 1 g of tissue. Notably, for ingestible capsules, the input power of the antenna is generally limited to 25 μW (−16 dBm) as per ITU-R RS.1346 guidelines, which is substantially lower than the maximum allowable input power. This calculative framework satisfactorily qualifies the proposed capsule device as suitable and safe for human beings.

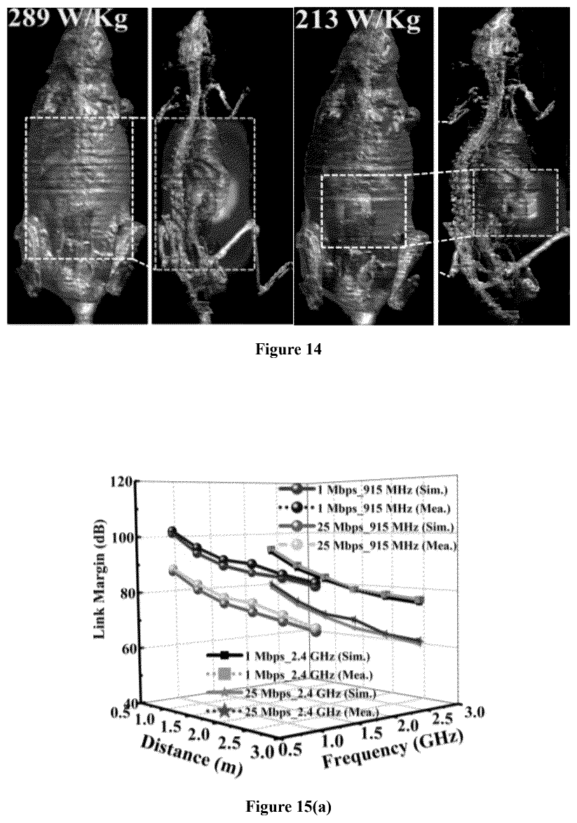

FIG. 14 illustrates Coronal and Sagittal view of averaged SAR distribution in anatomical rat model over 1 g of tissue standards.

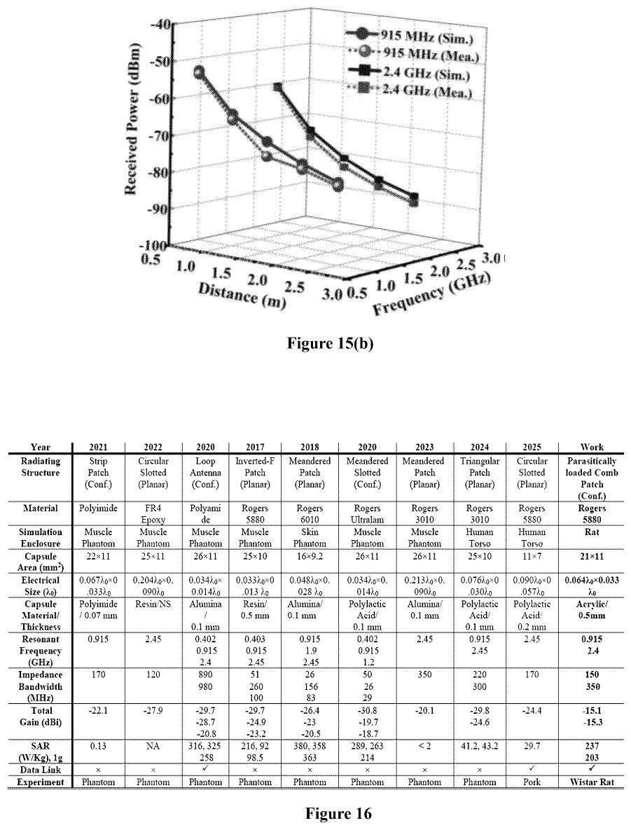

In an experiment, the efficacy of the proposed capsule for seamless wireless communication with an external monitor is analyzed by wireless monitoring of the body temperature of Wistar rat in real-time using temperature sensor DS18B20 and software-defined radio (SDR) module NI USRP B210. This marks a significant milestone and underscores the practical relevance of the study, addressing gaps overlooked in previous works. All animal experiments were conducted in compliance with ethical standards and government regulations, utilizing approved animal facilities under the supervision of qualified medical professionals. Now, to achieve the goal of transferring real-time data to an externally placed receiver, an experimental framework has been set up. The capsule antenna functions as the transmitter (Tx) and is implanted inside the abdomen of the rat, whereas, an omnidirectional monopole antenna of the SDR located in free-space serves as the receiver (Rx). Tx and Rx are connected to port-1 and port-2 of the SDR, respectively, where Tx is separated from the Rx by distance (d). Sensor DS18B20 is also inserted inside the rat's abdomen to collect the body temperature and is interfaced with the laptop using the Arduino Uno R3 SMD circuit module. The dedicated software in the laptop displays the collected 60 temperature values (in one minute) in the read buffer and transfers the temperature values to port-1 of the SDR module to enable the implanted capsule antenna to transmit the data wirelessly. To demonstrate uplink communication, a PSK-modulated signal is generated by the SDR module and transmitted via the proposed implanted capsule antenna. At both frequencies, 0.915 GHz and 2.4 GHz, the system is configured with a transmit power (Pt) of −16 dBm to comply with the ITU-R RS.1346 guidelines. These regulations also restrict the maximum allowed effective isotropic radiated power (EIRP) for implantable antennas to 36 dBm and 20 dBm at 0.915 GHz and 2.4 GHz, respectively, to prevent electromagnetic interference with nearby wireless devices operating in the same frequency bands. But, due to limited transmit gain (Gt) of −15.1 dBi (@0.915 Ghz) and −15.3 dBi (@2.4 Ghz), EIRP of the capsule antenna is fixed to −31.1 dBm and −31.3 dBm, respectively. Further, for WCE applications, the typical data transfer rate is 2 frames per second with a frame size of 256×256 pixels, requiring a minimum data rate of 1 Mb/s. Therefore, the omnidirectional monopole antenna having a gain (Gr) of 2.9 dBi receives the data packets at port-2 of the SDR module from ≈1 m distance when temperature values are sent at a bit rate of 1 Mbps and 25 Mbps at both frequencies. All the 60 temperature values can be seen as recovered messages in the display. Noted, before conducting measurement, the body temperature was 90.7° F. (32.6° C.) as per thermometer, but due to invasive procedure, the body temperature dropped to 29.25° C. (average of 60 readings) as read by the sensor. The recovered temperature was 28.69° C. (average of 60 readings). Further, the relation among data rates, frequencies, and distance for the proposed The link margin (LM) is determined, and the involved parameters are shown in Table IV. As per curve trajectories, the calculated LM shows its proximity to the measured LM, and 10-20 dB LM is considered enough for a seamless communication link. As the distance and frequency increase, the link margin decreases, which can be attributed to the increase in path loss. Additionally, at 0.915 Ghz, the link margin is better than that of the 2.4 GHz frequency band. Hence, it can be concluded that the proposed capsule antenna is capable of providing an LM greater than 60 dB up to 3 m distance for both 1 Mbps and 25 Mbps data rates at both frequencies. Furthermore, the comparison of calculated and measured received power (Pr) for a nominal telemetry distance of 0.5 m-2.5 m is shown in FIG. 15(b). The measured Pr is taken from the SDR module, and Pr is calculated.

FIG. 15 illustrates Simulated (calculated) and experimental (a) link budget, and (b) received power at different data rates with varying distances between Tx and Rx, at 0.915 GHz and 2.4 GHz. From FIG. 15(b), it is evident that the capsule antenna with transmit power (−16 dBm) can enable the monopole antenna with 2.9 dBi gain to receive more than −80 dBm power up to 2 m distance at both the ISM frequencies. The power levels are comparable because at higher frequencies, both the high radiation efficiency and path loss may get balanced. Although, during measurement, both monopole and capsule antenna used were linearly polarized, which sometimes could not maintain a reliable link, still, in different orientations (XZ, YZ, and XY plane) of the capsule, power levels are not significantly dropped in the two frequency bands due to the quasi-omnidirectional radiation patterns of the designed capsule. Finally, as the receiver sensitivity in the SDR module was −112 dBm, the given statistics indicate that the proposed capsule prototype has the potential to transmit a high-data-rate signal and handle seamless wireless communication even beyond a 3 m distance in WCE applications.

FIG. 16 illustrates a table depicting comparison of the proposed capsule antenna system with state-of-the-art;

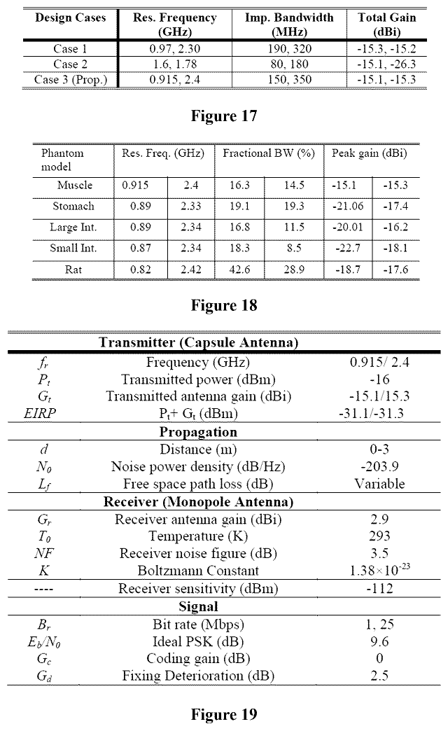

FIG. 17 illustrates a table depicting performance comparison of the three cases;

FIG. 18 illustrates a Table depicting simulated performance behavior on different phantoms models;

FIG. 19 illustrates a table depicting link budget parameters for the proposed capsule antenna;

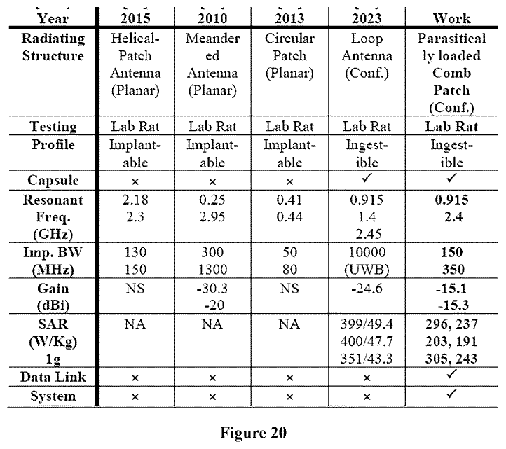

FIG. 20 illustrates a table depicting in-vivo experimental performance comparison on rat;

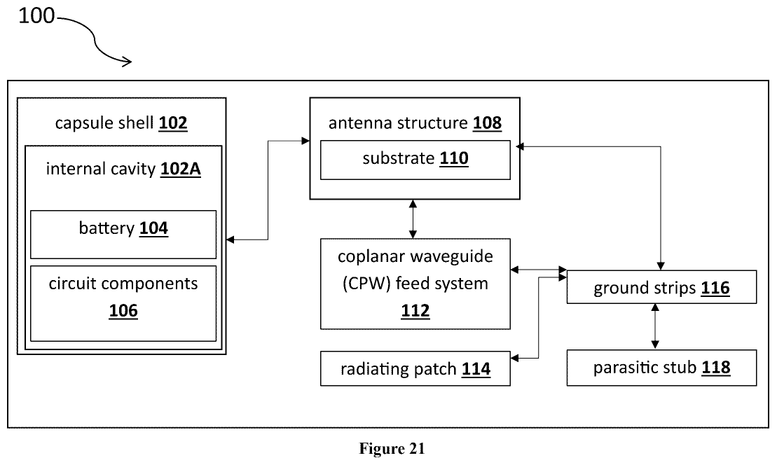

FIG. 21 illustrates a block diagram of a capsule antenna system in accordance with an embodiment of the present disclosure. The system includes a cylindrical capsule shell (102) fabricated of a biocompatible acrylic material having a thickness of 0.5 millimeter, a relative permittivity of 3.0, and a loss tangent of 0.001, the capsule shell (102) having an outer height of 21 millimeters and an outer diameter of 11 millimeters.

In an embodiment, an internal cavity (102A) within the capsule shell (102) is configured to house a battery (104) and circuit components (106), the battery (104) being modeled as a perfect electric conductor.

In an embodiment, a planar antenna structure (108) is fabricated on a substrate (110), optionally a Rogers 5880 substrate of dimensions 9 millimeters by 31 millimeters, the substrate (110) having a thickness of 0.254 millimeter, a relative permittivity of 2.2, and a loss tangent of 0.008.

The planar antenna structure (108) comprising a coplanar waveguide (CPW) feed system (112), a radiating patch (114), and ground strips (116) positioned on a same plane. The planar antenna structure (108) being wrapped onto an inner cylindrical surface of the capsule shell (102) to form a conformal antenna, wherein the substrate (110) acts as a dielectric shield between the radiating patch (114) and the battery (104).

In an embodiment, a multilayer dielectric configuration surrounding the antenna structure (108) comprising the substrate (110) as a first dielectric layer, the acrylic capsule shell (102) as a second dielectric layer.

In an embodiment, an external medium as a third dielectric layer, the combined layers defining an effective permittivity influencing current distribution and resonant behavior, wherein the radiating patch (114), the ground strips (116), and the feed system (112) are dimensioned and arranged such that current distribution along the antenna structure (108) follows half guided wavelength paths with patch currents traveling opposite to ground currents.

In another embodiment, the radiating patch (114) positioned above the ground strips (116) and coupled to the CPW feed system (112) comprises a rectangular patch having a length of 7.7 millimeters and a width of 31 millimeters, and wherein the ground strips (116) are coplanar and positioned parallel to opposite sides of the patch at a gap of 0.3 millimeters.

In a further embodiment, a first antenna configuration is formed by the rectangular patch with coplanar ground strips (116), the first configuration producing current paths equivalent to those of a folded half-wavelength dipole antenna, with a total path length of approximately 44.9 millimeters corresponding to half a guided wavelength.

In some embodiments, a second antenna configuration is formed by introducing notches into the ground strips (116) adjacent to a feed region and by extending the ground strips (116) toward curved edges of the substrate (110), the notched configuration increasing effective electrical length of surface current paths.

In one of the above embodiments, the rectangular patch is modified into a comb-shaped structure to cooperate with the notched ground strips (116), thereby defining multiple parallel conductive strips that establish additional current paths.

In some embodiments, a third antenna configuration is formed by introducing a parasitic stub (118) comprising an inverted comb-shaped strip positioned adjacent to and interlocked with the comb-shaped patch, the parasitic stub (118) being arranged to modify input impedance of the antenna structure (108).

In some embodiments, the parasitic stub (118) and the comb-shaped patch together define complementary conductive patterns that increase the radiation aperture area of the conformal antenna.

In some embodiments, surface current distributions are analyzed in planar configuration prior to conformal wrapping, and wherein after wrapping the antenna structure (108) conforms to the cylindrical curvature of the capsule shell (102) such that current path lengths are altered to approximately 83.6 millimeters and 56.4 millimeters in different modes, each corresponding to half guided wavelengths.

In some embodiments, the multilayer dielectric configuration comprises a first layer of substrate (110) of thickness 0.254 millimeter, a second layer of acrylic capsule wall of thickness 0.5 millimeter, and an external tissue-mimicking medium modeled as a homogeneous phantom having dielectric permittivity of 54.9 and conductivity of 0.948 siemens per meter, the combined thickness of the substrate (110) and capsule wall being 0.754 millimeter.

In some embodiments, the conformal wrapping angle of the planar antenna structure (108) alters the degree of field coupling between comb-shaped patch strips, parasitic stub strips, and ground strips (116), thereby changing effective current path lengths.

In some embodiments, the multilayer dielectric configuration is modeled as comprising three distinct dielectric regions, namely the substrate (110), the acrylic capsule wall, and an external surrounding medium, the effective permittivity being greater than the substrate permittivity due to fringing fields across the layers.

In a further embodiment, the system further comprising an optional shielding layer positioned between the wrapped antenna structure (108) and the packaged battery (104), the shielding layer having a height of 9 millimeters and a diameter of 7 millimeters; and wherein the shielding layer comprises a dielectric polyimide material of thickness 0.05 millimeter, relative permittivity 4.3, and loss tangent 0.008; and wherein the shielding layer comprises a metallic perfect electric conductor sheet of thickness 0.03 millimeter.

In some embodiments, the substrate (110) of the antenna structure (108) performs a dual role as both a supporting dielectric for the radiating patch (114) and as an intrinsic shielding material separating the antenna from the packaged battery (104); and wherein the capsule shell (102) is fabricated of an acrylic material selected to be biocompatible and to function as a superstrate layer contributing to the multilayer dielectric stack surrounding the antenna.

In some embodiments, the acrylic capsule wall serving as a superstrate prevents electrical shorting between the antenna structure (108) and conductive surroundings while simultaneously influencing fringing fields.

In some embodiments, the current distribution in the rectangular patch with coplanar ground strips (116) originates at the feed point and returns to the feed point along a path length equivalent to half a guided wavelength.

In some embodiments, the notched ground strips (116) adjacent to the feed region establish an extended surface current path along a perimeter of the ground strips (116), thereby enabling multiple resonant modes within the same conformal structure.

In some embodiments, the conformal antenna operates in a transmission line mode and an antenna mode, each mode corresponding to distinct current path lengths measured as half guided wavelengths; wherein the current path lengths in the conformal configuration include a longer path of approximately 83.6 millimeters corresponding to a first resonant mode and a shorter path of approximately 56.4 millimeters corresponding to a second resonant mode.

In some embodiments, variation in bending angle of the conformal wrapping modifies electromagnetic field coupling between the comb-shaped patch, the parasitic stub (118), and the notched ground strips (116), thereby altering the resonant mode supported by the antenna.

In some embodiments, the parasitic stub (118) positioned adjacent to the radiating patch (114) having a meandering structure comprising a plurality of conductive segments connected in a serpentine pattern, wherein the radiating patch (114) having a meandering structure comprising a plurality of conductive segments connected in a serpentine pattern, wherein the meandering structure of the radiating patch (114) and the parasitic patch have substantially identical shapes and arranged in a parallel configuration.

In some embodiments, the antenna system is configured to operate at a first frequency band centered around 0.97 GHz with an impedance bandwidth of approximately 150 Mhz, and a second frequency band centered around 2.4 GHz with an impedance bandwidth of approximately 350 MHz.

In some embodiments, the system (100) further comprising a temperature monitoring sensor integrated with the circuit components (106), configured to collect temperature data from within a digestive tract.

In some embodiments, the CPW feed system (112) comprising two feed lines extending from opposite sides of the radiating patch (114), wherein the CPW feed line positioned to facilitate directional current flow from the ground strips (116) toward the interdigital radiating patch (114) and the interdigital radiating patch (114) configured to distribute current through an elongated path along the CPW feed line, wherein the current flow exhibits two distinct minima points, one at the edges of the ground strips (116) and the other near the edges of the patch, wherein separation between the two distinct minima points corresponds to approximately half the guided wavelength.

In some embodiments, the cylindrical capsule shell (102) includes a coaxial cable connectivity port integrated through a drilled hole in the capsule cap for antenna connectivity.

In an embodiment, the planar antenna structure formed on the dielectric substrate is configured such that the radiating patch, the ground strips, and the coplanar feed lines are fabricated as an integrated metallization pattern, the pattern being continuous across the substrate surface and rolled into a cylindrical configuration along the inner surface of the capsule shell, the rolling of the substrate being arranged to maintain alignment of the patch and ground strips with the longitudinal axis of the capsule, thereby establishing a conformal geometry that preserves electrical continuity of the patterned conductors during the transition from planar to cylindrical form.

In an embodiment, the coplanar ground strips extend longitudinally on opposite sides of the radiating patch and are configured to form a symmetrical current return path, the ground strips being arranged such that surface currents traveling along the patch encounter equal and opposite return currents on the ground strips, and wherein the coplanar feed line is positioned to provide a direct electrical transition between the ground strips and the feed pad of the radiating patch, the arrangement maintaining balanced electromagnetic coupling across the width of the antenna structure.

In an embodiment, the radiating patch comprises a segmented conductive structure divided into multiple parallel strip elements separated by slots to define a comb-shaped arrangement, the slots extending across the length of the radiating patch to increase the overall electrical path length available for surface currents, the comb-shaped configuration being further arranged such that the parallel strip elements of the patch cooperate with the adjacent ground strips to support multiple parallel current paths distributed across the substrate surface.

In an embodiment, a parasitic stub structure is disposed adjacent to the segmented radiating patch, the parasitic stub comprising conductive strips arranged in a pattern that interdigitates with the comb-shaped strip elements of the radiating patch, the interdigitated arrangement forming a lock-and-key structural relationship between the parasitic stub and the radiating patch, the arrangement being configured to establish electromagnetic coupling between the parasitic stub and the radiating patch so as to modify current flow distribution without direct electrical connection.

In an embodiment, the multilayer dielectric configuration is concentrically arranged such that the antenna substrate forms an inner dielectric layer, the capsule wall forms an intermediate dielectric layer, and the external surrounding medium forms an outer dielectric layer, the concentric arrangement enclosing the antenna structure completely along its radial thickness, the capsule wall thereby simultaneously performing the dual roles of providing structural integrity to the capsule assembly and acting as a dielectric superstrate positioned over the antenna conductors to prevent electrical contact with external conductive environments.

A conformal parasitically loaded comb shaped coplanar antenna integrated within a compact ingestible capsule endoscopy system is designed and experimentally validated through in vivo measurements conducted on Wistar rats. The capsule system is capable of monitoring the body temperature of the Wistar rat wirelessly in dual ISM bands of frequency 0.915 and 2.4 GHz, with impedance bandwidths of 150 MHz and 350 MHz, respectively. Before conducting the measurement, the body temperature of the subject was 90.7° F. (32.6° C.) as per the thermometer, but, due to the invasive procedure, the body temperature dropped to 29.25° C. as read by the sensor, and the recovered temperature was 28.69° C. From EMI evaluation, it is observed that in the proposed capsule design, having an extra dielectric layer can be useful as a shield because it confines the field distribution to the lesser area on the battery as compared to the case without any shield. Further, the simulation is validated by measuring the proposed dual-band capsule antenna system in different environments (muscle phantom, minced pork, and Wistar rat). In order to comply with FCC and ICNIRP guidelines, the highest permissible input power to the capsule antenna must not exceed 0.0052 W (7.16 dBm) and 0.0065 W (8.13 dBm) for 1 g of tissues. The capsule antenna with transmit power (−16 dBm) can also enable the receiver monopole antenna with 2.9 dBi gain to receive more than −80 dBm power up to 2 m distance at both the ISM frequencies. The satisfactory in-line relation of the measured and simulated results shows the reasonable proof of concept that the proposed capsule is a potential candidate for wireless endoscopy applications. Finally, Table-V further highlights the novelty by comparing our proposed ingestible capsule-based in-vivo study using Wistar rat with the existing in-vivo studies.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

We claim:

1. A capsule antenna system comprising:
a cylindrical capsule shell fabricated of a biocompatible acrylic material having a thickness of 0.5 millimeter, a relative permittivity of 3.0, and a loss tangent of 0.001, the cylindrical capsule shell having an outer height of 21 millimeters and an outer diameter of 11 millimeters;
an internal cavity within the cylindrical capsule shell configured to house a battery and circuit components, the battery being modeled as a perfect electric conductor;
a planar antenna structure fabricated on a substrate of dimensions 9 millimeters by 31 millimeters, the substrate having a thickness of 0.254 millimeter, a relative permittivity of 2.2, and a loss tangent of 0.008;
the planar antenna structure comprising a coplanar waveguide (CPW) feed system, a radiating patch, and ground strips positioned on a same plane;
the planar antenna structure being wrapped onto an inner cylindrical surface of the cylindrical capsule shell to form a conformal antenna, wherein the substrate acts as a dielectric shield between the radiating patch and the battery;
a multilayer dielectric configuration surrounding the planar antenna structure comprising the substrate as a first dielectric layer, an acrylic capsule shell as a second dielectric layer, and an external medium as a third dielectric layer, a plurality of combined layers defining an effective permittivity influencing current distribution and resonant behavior; wherein the radiating patch, the ground strips, and the feed system are dimensioned and arranged such that current distribution along the planar antenna structure follows half guided wavelength paths with patch currents traveling opposite to ground currents; wherein the radiating patch positioned above the ground strips and coupled to the CPW feed system comprises a rectangular patch having a length of 7.7 millimeters and a width of 31 millimeters, and wherein the ground strips are coplanar and positioned parallel to opposite sides of the patch at a gap of 0.3 millimeters; and wherein a first antenna configuration is formed by the rectangular patch with coplanar ground strips, the first antenna configuration producing current paths equivalent to those of a folded half-wavelength dipole antenna, with a total path length of approximately 44.9 millimeters corresponding to half a guided wavelength; and wherein the current distribution in the rectangular patch with coplanar ground strips originates at a feed point and returns to the feed point along a path length equivalent to half a guided wavelength; and wherein the coplanar ground strips extend longitudinally on opposite sides of the radiating patch and are configured to form a symmetrical current return path, the ground strips being arranged such that surface currents traveling along a patch encounter equal and opposite return currents on the ground strips, and wherein a coplanar feed line is positioned to provide a direct electrical transition between the ground strips and a feed pad of the radiating patch, the arrangement maintaining balanced electromagnetic coupling across the width of the planar antenna structure.

2. The capsule antenna system of claim 1, wherein a second antenna configuration is formed by introducing notches into the ground strips adjacent to a feed region and by extending the ground strips toward curved edges of the substrate, the notched configuration increasing effective electrical length of surface current paths, and wherein the ground strips notched adjacent to the feed region establish an extended surface current path along a perimeter of the ground strips, thereby enabling multiple resonant modes within same conformal structure.

3. The capsule antenna system of claim 2, wherein the rectangular patch is modified into a comb-shaped structure to cooperate with notched ground strips, thereby defining multiple parallel conductive strips that establish additional current paths; and wherein a third antenna configuration is formed by introducing a parasitic stub comprising an inverted comb-shaped strip positioned adjacent to and interlocked with a comb-shaped patch, the parasitic stub being arranged to modify input impedance of the planar antenna structure.

4. The capsule antenna system of claim 3, wherein the parasitic stub and the comb-shaped patch together define complementary conductive patterns that increase a radiation aperture area of the conformal antenna; and wherein a conformal wrapping angle of the planar antenna structure alters a degree of field coupling between comb-shaped patch strips, parasitic stub strips, and ground strips, thereby changing effective current path lengths; and; and wherein variation in a bending angle of the conformal wrapping modifies electromagnetic field coupling between the comb-shaped patch, the parasitic stub, and the notched ground strips, thereby altering the resonant modes supported by the third antenna.

5. The capsule antenna system of claim 4, wherein the parasitic stub positioned adjacent to the radiating patch having a meandering structure comprising a plurality of conductive segments connected in a serpentine pattern, wherein the radiating patch having a meandering structure comprising a plurality of conductive segments connected in a serpentine pattern, wherein the meandering structure of the radiating patch and a parasitic patch have substantially identical shapes and arranged in a parallel configuration.

6. The capsule antenna system of claim 1, wherein surface current distributions are analyzed in planar configuration prior to conformal wrapping, and wherein after wrapping the planar antenna structure conforms to a cylindrical curvature of the cylindrical capsule shell such that current path lengths are altered to approximately 83.6 millimeters and 56.4 millimeters in different modes, each corresponding to half guided wavelengths; and wherein the multilayer dielectric configuration comprises a first layer of substrate of thickness 0.254 millimeter, a second layer of acrylic capsule wall of thickness 0.5 millimeter, and an external tissue-mimicking medium comprising a homogeneous phantom having dielectric permittivity of 54.9 and conductivity of 0.948 siemens per meter, a combined thickness of the substrate and capsule wall being 0.754 millimeter.

7. The capsule antenna system of claim 1, wherein the multilayer dielectric configuration comprises three distinct dielectric regions, namely the substrate, an acrylic capsule wall, and an external surrounding medium, the effective permittivity being greater than a substrate permittivity due to fringing fields across the first dielectric layer.

8. The capsule antenna system of claim 1, further comprising an shielding layer positioned between a wrapped antenna structure and a packaged battery, the shielding layer having a height of 9 millimeters and a diameter of 7 millimeters; and wherein the shielding layer comprises a dielectric polyimide material of thickness 0.05 millimeter, relative permittivity 4.3, and loss tangent 0.008; and wherein the shielding layer comprises a metallic perfect electric conductor sheet of thickness 0.03 millimeter.

9. The capsule antenna system of claim 1, wherein the substrate of the planar antenna structure performs a dual role as both a supporting dielectric for the radiating patch and as an intrinsic shielding material separating the capsule antenna system from a packaged battery; and wherein the cylindrical capsule shell is fabricated of an acrylic material selected to be biocompatible and to function as a superstrate layer contributing to a multilayer dielectric stack surrounding the capsule antenna system; and wherein an acrylic capsule wall serving as a superstrate prevents electrical shorting between the planar antenna structure and conductive surroundings while simultaneously influencing fringing fields.

10. The capsule antenna system of claim 1, wherein the conformal antenna operates in a transmission line mode and an antenna mode, each mode corresponding to distinct current path lengths measured as half guided wavelengths; wherein the distinct current path lengths in a conformal configuration include a longer path of approximately 83.6 millimeters corresponding to a first resonant mode and a shorter path of approximately 56.4 millimeters corresponding to a second resonant mode.

11. The capsule antenna system of claim 1, wherein the capsule antenna system is configured to operate at a first frequency band centered around 0.97 GHz with an impedance bandwidth of approximately 150 MHz, and a second frequency band centered around 2.4 GHz with an impedance bandwidth of approximately 350 MHz; and wherein the cylindrical capsule shell includes a coaxial cable connectivity port integrated through a drilled hole in the capsule antenna system for antenna connectivity.

12. The capsule antenna system of claim 1, further comprising a temperature monitoring sensor integrated with the circuit components, configured to collect temperature data from within a digestive tract; and wherein the CPW feed system comprising two feed lines extending from opposite sides of the radiating patch, wherein a CPW feed line positioned to facilitate directional current flow from the ground strips toward an interdigital radiating patch and the interdigital radiating patch configured to distribute current through an elongated path along the CPW feed line, wherein the directional current flow exhibits two distinct minima points, one at edges of the ground strips and other near the edges of the patch, wherein separation between the two distinct minima points corresponds to approximately half the guided wavelength.

13. The capsule antenna system of claim 1, wherein the planar antenna structure formed on a dielectric substrate is configured such that the radiating patch, the ground strips, and the coplanar feed line is fabricated as an integrated metallization pattern, the integrated metallization pattern being continuous across a substrate surface and rolled into a cylindrical configuration along the inner cylindrical surface of the cylindrical capsule shell, a rolling of the substrate being arranged to maintain alignment of the patch and ground strips with a longitudinal axis of the capsule antenna system, thereby establishing a conformal geometry that preserves electrical continuity of patterned conductors during the direct electrical transition from planar to cylindrical form.

14. The capsule antenna system of claim 1, wherein the radiating patch comprises a segmented conductive structure divided into multiple parallel strip elements separated by slots to define a comb-shaped arrangement, the slots extending across the length of the radiating patch to increase an overall electrical path length available for surface currents, a comb-shaped configuration being further arranged such that the parallel strip elements of the patch cooperate with adjacent ground strips to support multiple parallel current paths distributed across a surface of the substrate

15. The capsule antenna system of claim 1, wherein a parasitic stub structure is disposed adjacent to a segmented radiating patch, the parasitic stub structure comprising conductive strips arranged in a pattern that interdigitates with a comb-shaped strip elements of a radiating patch, the interdigitated arrangement forming a lock-and-key structural relationship between the parasitic stub structure and the radiating patch, an arrangement being configured to establish electromagnetic coupling between the parasitic stub structure and the radiating patch so as to modify current flow distribution without direct electrical connection.

16. The capsule antenna system of claim 1, wherein the multilayer dielectric configuration is concentrically arranged such that an antenna substrate forms an inner dielectric layer, a capsule wall forms an intermediate dielectric layer, and an external surrounding medium forms an outer dielectric layer, the concentric arrangement enclosing the planar antenna structure completely along its radial thickness, the capsule wall thereby simultaneously performing dual roles of providing structural integrity to the capsule wall and acting as a dielectric superstrate positioned over antenna conductors to prevent electrical contact with external conductive environments.
